(19) United States
(12) Patent Application Publication
(10) Pub. No.: US 2005/0234123 A1
(43) Pub. Date: Oct. 20, 2005

(54) Process

(76) Inventors: Aldo Belli, Cornate d' Adda (IT); Vincenzo Cannata, Sasso Marconi (IT); Telly Fonduca, Sodertalje (SE); Martin Hedberg, Sodertalje (SE); Andreas Westermark, Sodertalje (SE); Marco Villa, Padova (IT)

Correspondence Address: WHITE & CASE LLP;PATENT DEPARTMENT, 1155 AVENUE OF THE AMERICAS, NEW YORK, NY, 10036, US

(21) Appl. No.: 10/497,012

(22) PCT Filed: Nov. 26, 2002
(86) PCT No.: PCT/SE02/02184

§ 371 Date: Jun. 9, 2005

(30) Foreign Application Priority Data
Nov. 27, 2001 (SE) 0103978-3

(51) Int. Cl. A61K 31/21; C07C 23/02

(52) U.S. Cl. 514/509; 558/482

(57) ABSTRACT

At the present invention relates to a new process for the preparation of the (S)-naproxen 4-nitrooxybutyl ester and to new intermediates obtained and used therein. The invention further relates to the use of the new intermediates for the manufacturing of pharmaceutically active compounds such as (S)-naproxen 4-nitrooxybutyl ester. The invention also relates to the use of (S)-naproxen 4-nitrooxybutyl ester prepared according to the process of the present invention for the manufacturing of a medicament for the treatment of pain.

### FIELD OF THE INVENTION

The present invention relates to a new process for the preparation of the 4-nitrooxybutyl ester of 2-(S)-(6-methoxy-2-naphtyl)-propanoic acid (herein after named (S)-naproxen) and to new intermediates prepared therein suitable for large scale manufacturing of (S)-naproxen. The invention further relates to the use of the new intermediates for the manufacturing of pharmaceutically active compounds such as (S)-naproxen 4-nitrooxybutyl ester.

### BACKGROUND OF THE INVENTION

(S)-Naproxen 4-nitrooxybutyl ester is known for its pharmaceutical activity as an antiinflammatory and/or analgesic agent. The advantages of (S)-naproxen 4-nitrooxybutyl ester compared to (S)-naproxen are among others a good tolerance and the reduction of gastrointestinal side effects.

Different processes for the preparation of (S)-naproxen 4-nitrooxybutyl ester have been described in the prior art.

WO 01/10814 discloses a process for the preparation of (S)-naproxen 4-nitrooxyalkyl ester with an optical purity of 97%. In said process an acid halide of (S)-naproxen is reacted with a nitrooxyalkanol in an inert organic solvent such as dichloromethane, chlorobenzene, xylene or toluene, to give a (S)-naproxen 4-nitrooxyalkyl ester. In the process of the present invention the use of potentially explosive intermediates such as nitrooxyalkanols as described in WO 01/10814 is avoided.

In U.S. Pat. No. 5,703,073 the preparation of a (O)-nitrosylated ester of (S)-naproxen is performed by reacting the acid chloride of (S)-naproxen with 1,4-butanediol to form a 4-hydroxybutyl ester of (S)-naproxen, which is then reacted with nitrosonium tetrafluoroborate in an anhydrous solvent (dichloromethane) to form (S)naproxen 4-(nitrosooxy)butyl ester.

WO 95/09831 describes a process whereby the sodium salt of (S)-naproxen is reacted with a halo-butanol such as 4-bromobutan-1-ol or 4-chlorobutan-1-ol. The ester is then halogenated in the presence of PBr₃ and the like. Alternatively, the monomeric ester is formed by reacting the sodium salt derivative with a 1,4-dihalobutane. The monomeric ester with the terminal halogen is then reacted with a nitrate source such as silver nitrate. The process may be performed in solvents such as chloroform, 1,4-dioxane, tetrahydrofuran and the like. The use of silver nitrate to achieve a good yield of the product constitutes an economical drawback for large scale manufacturing of (S)-naproxen 4-nitrooxybutyl ester.

The process of the present invention uses a sulfonated intermediate. This intermediate can be easily manufactured and is highly reactive for reactions with nitrate ions to form (S)-naproxen 4-nitrooxybutyl ester. Nitrate substitution of sulfonates has been described in the literature.

In Cainelli, et al. (Tetrahedron Lett., 1985, 28, 3369-3372) and Cainelli, et al. (Tetrahedron 1985, 41, 1385-1392), the substitution of sulfonate esters with tetrabutylammonium nitrate or an ion-exchanger with nitrate ions in a solvent such as pentane, toluene or benzene, is described. During this process high temperatures are used, which makes the process unsafe to use for large scale production.

Cainelli, et al. (J. Chem. Soc. Perkin Trans. I, 1987, 2637-2642) describe the nitrate substitution of sulfonate esters by reacting alkylmethanesulfonates with tetrabutylammonium nitrate in toluene.

In Kawamura, et al. (Chem. Parm. Bull., 1990, 38, 2092-2096) an alkylphenylsulfonate is reacted with tetrabutylammonium nitrate in toluene.

The costs for the tetraalkylammonium nitrate sources used in stoichiometric amounts as described in these prior art documents are economically undesirable for large-scale manufacturing of (S)-naproxen 4-nitrooxybutyl ester. Processes wherein cheaper and low molecular weight alkali metal nitrates can be used are preferred for economical reasons. However, tetraalkylammonium nitrates may be used as phase transfer catalysts in substoichiometric amounts.

In Hwu, et al. (Synthesis, 1994, 471-474) the preparation of nitrate esters from sulfonic acid esters is described. The rather high temperatures and long reaction times used in l0 combination with the low stability of the end products obtained, makes this process less suitable for large-scale production. In addition, the molar excess of sodium nitrate is at least twice as large as in the present invention, which increases costs and may give more waste problems. Further, the crude product obtained by the method according to Hwu et al, needs to be purified either by way of chromatography or distillation to obtain a pharmaceutically acceptable purity. Neither of these two purification options are appreciated for the large scale manufacturing of (S)naproxen 4-(nitrooxy)butyl ester.

ES 2,073,995, discloses the syntheses of alkyl nitrate esters from alkylsulfonates or 4-toluenesulfonates and metal nitrates using solvents such as dimethyl formamide, dimethyl acetamide, acetonitrile or dimethylsulfoxide. Using dimethyl acetamide or dimethylsulfoxide as solvent in the synthesis of (S)-naproxen 4-(nitrooxy)butyl ester starting from for instance (S)-naproxen 4-(methanesulfonyloxy)butyl ester gives a crude product which needs to be purified either by chromatography or by distillation to achieve a pharmaceutically acceptable quality.

In summary, there is a need for a more convenient and more economically efficient process for the manufacturing of liquid state bulk products of pharmaceutical quality, like (S)-naproxen 4-nitrooxybutyl ester and intermediates like (S)-naproxen 4-(sulfonyloxy)butyl ester, especially with regard to large-scale production where factors like costs, manufacturing time, use of more environmentally friendly solvents, etcetera are vital for commercial application. The present invention provides for such a process.

### DETAILED DESCRIPTION OF THE INVENTION

The present invention provides for a new process to prepare (S)-naproxen 4-nitrooxybutyl ester. Further, it provides for a new process to prepare compounds, which are useful as intermediates in the preparation of pharmaceutically active compounds such as (S)-naproxen 4-nitrooxybutyl ester, especially with regard to large-scale manufacturing of said ester.

The new synthetic route for the manufacture of (S)-naproxen 4-nitrooxybutyl ester is described below in two alternative routes, steps 1a and 2 and steps 1b, 1c and 2.

In step 1 of the manufacturing process a compound of formula I 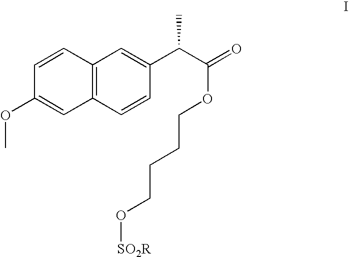
is prepared in reaction step 1a or alternatively in a two step process of steps 1b and 1c.

In step 1a (S)-naproxen, the acid halide or the salt derivative of (S)-naproxen 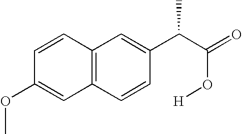
is reacted with a compound of formula II 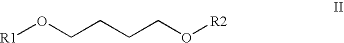
wherein R1 is H or RSO₂ and R2 is RSO₂ and
R is C₁-C₄ alkyl, C₁-C₄ alkylphenyl, phenyl, phenylmethyl, halophenyl, nitrophenyl, halogen, CF₃ or n-C₄F₉, and the halo is fluoro, chloro or bromo, to obtain the compound of formula I.
i) In the case of R1═R2═RSO₂, the reaction can be performed in a known manner described in the prior art, e.g. using (S)-naproxen sodium salt as starting material, which is first prepared from (S)-naproxen. This salt may then be reacted with the compound of formula II, wherein R1═R2═RSO₂ using conditions similar to those described in the prior art (WO95/09831).
iia) In the case of R1═H and R2═RSO₂, the compound of formula I can be prepared by reacting the compound of formula II, wherein R1═H and R2═RSO₂ with (S)-naproxen under acid catalysis in the same manner as described for compound of formula III below.
iib) Alternatively, (S)-naproxen may first be converted to its corresponding acid halide as described in WO 01/10814. The acid chloride of (S)-naproxen may then be reacted with the compound of formula II, wherein R1═H and R2═RSO₂, using a base such as pyridine or potassium carbonate to give the compound of formula I.

In another alternative, the compound of formula I may be prepared in a two step process, whereby in
step 1b, a compound of formula III is obtained by reacting (S)-naproxen with the compound of formula II, whereby R1 and R2 are both H, 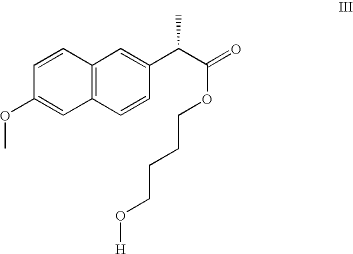
   followed by
step 1c, wherein the compound of formula III may be reacted with RSO₂Cl and R is C₁-C₄ alkyl, C₁-C₄ alkylphenyl, phenyl, phenylmethyl, halophenyl, nitrophenyl, halogen, CF₃ or n-C₄F₉, and the halo is fluoro, chloro or bromo, to give the compound of formula I.

The esterification step 1b can be performed in a manner known to a person skilled in the art, for example by treating (S)-naproxen and the 1,4-butanediol (compound II) with an acidic or dehydrating agent selected from the group consisting of sulphuric acid or its salts, perchloric acid (e.g. 70%) or other suitable acids such as polystyrene sulphonic acids, zeolites, acidic clays, sand in combination with strong hydrophilic acids such as perchloric acid or gaseous hydrogen chloride and montmorillonites.

The acids may be used in the gas, fluid or solid form. The solid heterogeneous acids can relatively easily be filtered from the reaction solution and re-used in large-scale production processes.

Examples of other coupling reagents useful for the esterification step 1b are carbodiimides such as N,N′-dicyclohexylcarbodiiniide (DCC), acid chlorides such as oxalyl chloride, chloroformates such as isobutyl chloroformate or other reagents such as cyanuric chloride, N,N′-carbonyldiimidazole, diethyl chlorophosphite, 2-chloro-1-methyl-pyridinium iodide and 2,2′-dipyridyl disulphide.

The reaction step 1b may be performed in a solvent selected from the group consisting of aromatic hydrocarbons such as benzene or toluene, aliphatic hydrocarbons such as n-heptane, ketones such as methyl isobutylketone, ethers such as tetrahydrofuran or diethyleneglycol dimethyl ether, chlorinated hydrocarbons such as dichloromethane or, chlorobenzene, or mixtures thereof.

Preferred solvents are non-polar and/or non acidic solvents.

Alternatively, an excess of 1,4-butanediol may be used as solvent optionally mixed with any of the other organic solvents mentioned above.

The total amount of solvents used in the esterification process step 1b, may vary between 0 to 100 volume parts per weight of starting material.

The temperature of the esterification step 1b may be between −100═ C. to +130° C., preferably between 0° C. and +120° C.

Compounds of formula III as obtained in step 1b may be purified by way of a two-way extraction to obtain a solution comprising the compound of formula III having a chromatographic purity of at least 95% and prefarbly more than 97% (extraxtion step i) and a 1,4-butandiol content below about 0.2% (w/w) (extraction step ii). The extractive purification may be done batch-wise or continuously.

Extraction Step i)

In this reaction step the chromatographic purity is improved. The solution used in this extraction step may comprise a mixture of i) 1,4-butanediol, ii) water and/or a low molecular weight aliphatic alcohol and iii) a hydrocarbon solvent or mixtures of organic solvents with hydrocarbon solvents.

The low molecular weight aliphatic alcohols may be selected from the group consisting of methanol, ethanol, propanol or mixtures thereof.

The hydrocarbon solvents may be selected from the group comprising of toluene, cumene, xylenes, ligroin, petroleum ether, halobenzenes, heptanes, hexanes, octanes, cyclohexanes, cycloheptanes and the like.

A suitable organic solvent may be selected from the groups consisting of ketones such as methyl iso-butyl ketone, ethers such as di-n-butyl ether, tert-butyl methyl ether and aliphatic esters such as ethyl acetate and n-butyl acetate, haloalkanes like dichloromethane or mixtures thereof.

The purified compound of formula III is obtained as a solution in a mixture of 1,4 butanediol, water and/or a low molecular weight aliphatic alcohol.

Extraction Step ii)

The extraction is performed to lower the 1,4-butanediol-content and performed after extraction step i) wherein the chromatographic purity is improved as described above. The solution may comprise i) a mixture of water and/or a low molecular weight aliphatic alcohol and ii) an organic solvent or mixtures of organic solvents.

The low molecular weight aliphatic alcohols may be selected from the group consisting of methanol, ethanol, propanol, or mixtures thereof.

A suitable organic solvent may be selected from the groups consisting of aromatic hydrocarbons such as toluene, cumene, xylenes, ketones such as methyl iso-butyl ketone, ethers such as di-n-butyl ether, tert-butyl methyl ether and aliphatic esters such as ethyl acetate and n-butyl acetate, haloalkanes like dichloromethane or mixtures thereof.

The purified compound of formula III having a chromatographic purity of at least 95% and preferably at least 97% and a 1,4-butanediol content below about 0.2% (w/w) is obtained as a solution in the organic solvent system and may then optionally be isolated by removal of the volatile solvents by vacuum distillation.

The reaction condition in step 1c, would suitably involve an excess of RSO₂Cl in an organic solvent or a mixture of organic solvents.

A suitable solvent in step 1c may be selected from the groups consisting of aromatic hydrocarbons such as toluene, cumene, xylenes, ketones such as methyl iso-butyl ketone, ethers such as di-n-butyl ether, tert-butyl methyl ether and tetrahydroffiran, aliphatic nitrites such as acetonitrile and aliphatic esters such as ethyl acetate and n-butyl acetate, haloalkanes like dichloromethane, or mixtures thereof.

Preferred solvents in step 1c are toluene, xylenes, ethyl acetate, acetonitrile, butyl acetate and isopropyl acetate.

A base may be added in step 1c. The base may be selected from the group consisting of triethylamine, pyridine, N-methylmorpholine, diisopropylethylamine, tributylamine and N-methyl-piperidine. The preferred bases are triethylamine and N-methylmorpholine.

Optionally a catalyst such as 4-(dimethylamino)pyridine may be used in step 1c.

Compounds of formula I as obtained in step 1c may be purified by crystallisation from an organic solvent, optionally using a hydrocarbon as antisolvent to obtain a crystalline solid having a purity of about 95% and preferably about 97% and an optical purity equal or better than the (S)-naproxen used as starting material for step 1b.

Suitable solvents used for the crystallisation may be selected from the group consisting of aromatic hydrocarbons such as toluene, cumene, xylenes, ketones such as methyl iso-butyl ketone, ethers such as di-n-butyl ether, tert-butyl methyl ether and tetrahydrofuran, aliphatic nitrites such as acetonitrile and aliphatic esters such as ethyl acetate and butyl acetate, or mixtures thereof.

Preferred solvents in step 1c are toluene, xylenes, ethyl acetate, acetonitrile, butyl acetate and isopropyl acetate.

As a suitable antisolvent for the crystallisation may be used toluene, cumene, xylenes, ligroin, petroleum ether, halobenzenes, heptanes, hexanes, octanes, cyclohexanes, cycloheptanes and the like.

In step 2 of the manufacturing process, (S)-naproxen 4-nitrooxybutyl ester (formula IV) 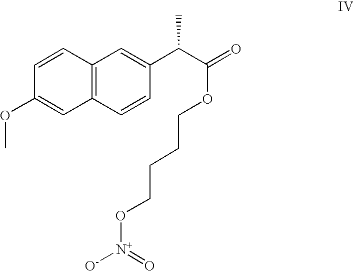
is obtained by reacting the compound of formula I with a nitrate source optionally in the presence of a solvent.

This reaction can be performed with a nitrate source selected from the group consisting of lithium nitrate, sodium nitrate, potassium nitrate, magnesium nitrate, calcium nitrate, iron nitrate, zink nitrate or tetraalkylammonium nitrate (wherein alkyl is a C₁-C₁₈-alkyl, which may be straight or branched).

Preferred nitrate sources may be selected from the group consisting of lithium nitrate, sodium nitrate, potassium nitrate, magnesium nitrate and calcium nitrate.

A suitable organic solvent in step 2 is preferably a polar aprotic solvents which may be selected from the group consisting of N-methylpyrrolidinone, N,N-dimethylacetamide, sulpholane, tetramethylurea or 1,3-dimethyl-2-imidazolidinone or nitriles such as acetonitrile, or mixtures thereof.

Other solvents may be aromatic hydrocarbons such as toluene, aliphatic hydrocarbons such as n-heptane, ketones such as methyl ethyl ketone, methyl isobutylketone, ethers such as tetrahydrofuran or diethyleneglycol dimethyl ether, chlorinated hydrocarbons such as chlorobenzene, aliphatic esters such as ethyl acetate, butyl acetate or isopropyl acetate, nitrated hydrocarbons such as nitromethane, ethylene glycols such as polyethylene glycol and mixtures of these, optionally with an added aliphatic alcohols such as methanol, ethanol, n-propanol, i-propanol, n-butanol, i-butanol or t-butanol.

The nitration step 2 may also be performed in water, optionally in combination with any of the above listed organic solvents.

The nitration step 2 may optionally be performed with a phase-transfer-catalyst. As a phase transfer-catalyst may be used tetraalkylammonium sali, arylalkylammonium salt, tetraalkylphosphonium salt, arylalkylphosphonium salt, crown ether or ethylene glycol such as pentaethylene glycol, hexaethylene glycol or polyethylene glycol, or mixtures thereof.

Further, a detergent may be used in the nitration step 2 to enhance the solubility of the starting material.

As a detergent may be used any commercially available non-ionic or ionic surfactant alone or in combinations. A non-ionic surfactant may be selected from the group consisting of sugar esters such as sorbitan monolaurate, sorbitan monooleate and polymeric surfactants such as polyoxyethylene sorbitan monostearate.

An ionic surfactant may be selected from the group consisting of glycolic acid ethoxylate alkyl ether, alkali metal alkyl 3-sulfopropyl ethers, sodium lauryl sulfate, sodium laureth sulfate, disodium laureth sulfonsuccinate, sodium stearate and cetyltrimethylammonium halides.

Thus, the nitration step 2 may be performed in water, optionally in combination with any of the above listed organic solvents and/or phase-transfer-catalysts and/or detergents.

The purity of the end product obtained in step 2 is preferably at least about 97%, particularly preferred at least about 98% and the optical purity is preferably similar or superior than the optical purity of the (S)-naproxen starting material.

The term “C₁-C₄ alkyl” means an alkyl having 1 to 4 carbon atoms and includes both straight and branched chain alkyl groups such as methyl, ethyl, n-propyl, i-propyl, n-butyl, i-butyl and t-butyl.

The term “C₁-C₄ alkylphenyl” means methylphenyl, ethylphenyl n-propylphenyl, i-propylphenyl, n-butylphenyl, i-butylphenyl and t-butylphenyl.

The term “phenylmethyl” means benzyl.

The term “halo” and “halogen” refer to fluoro, chloro or bromo.

The term “halophenyl” and “nitrophenyl” refer to phenyl groups substituted with one or more halogen or nitro group.

The term “large scale” means a manufacturing scale in the range of “kilogram to multiton”.

The temperature used in process step 1 and 2 may be between −100° C. to +130° C. The temperature is preferably kept below 130° C., because the stability of the end product might be affected by a high temperature. Particular preferred is a temperature between room temperature and 120° C.

Room temperature shall mean a temperature between 18° C. and 25° C.

Reaction step 2 is preferably performed at a temperature below 90° C.

The total amount of solvents may vary between 0 to 100 volume parts per weight of starting material.

The skilled person will appreciate that the different reaction steps need different reaction times.

In the process of the present invention the use of explosive intermediates such as nitrooxyalkanols are avoided. Furthermore, the new process is commercially and environmentally more advantageous than the known processes.

Another advantage of the process of the present invention is that the enantiomeric purity of the starting material is at least maintained in the end product.

A further object of the present invention is the compound of formula I 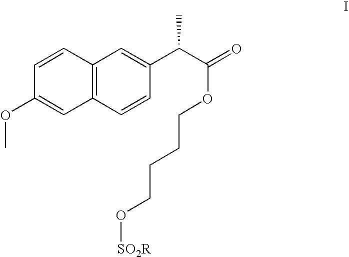
wherein R is phenylmethyl, halophenyl, nitrophenyl, halogen, CF₃ or n-C₄F₉ and the halo is fluoro, chloro or bromo.

Another object of the present invention relates to the use of the compound of formula I, wherein R is C₁-C₄ alkyl, phenyl, phenylmethyl, C₁-C₄ alkylphenyl, halophenyl, nitrophenyl, halogen, CF₃ or n-C₄F₉, and the halo is fluoro, chloro or bromo, as an intermediate for the manufacturing of a pharmaceutically active compound.

Yet another object of the present invention relates to the use of the compound of formula I, wherein R is C₁-C₄ alkyl, phenyl, phenylmethyl, C₁-C₄ alkylphenyl, halophenyl, nitrophenyl, halogen, CF₃ or n-C₄F₉ and the halo is fluoro, chloro or bromo, as an intermediate for the manufacturing of (S)-naproxen 4-nitrooxybutyl ester.

Yet another object of the present invention is the use of (S)-naproxen 4-nitrooxybutyl ester prepared according to the process described above under step 1 and 2, for the manufacturing of a medicament for the treatment of pain.

The present invention is described in more detail in the following non-limiting examples.

Preparation of the Intermediates.

### Synthesis of (S)-Naproxen 4-hydroxybutyl ester

### EXAMPLE 1

### (S)-Naproxen 4-hydroxybutyl Ester

A mixture of (S)-naproxen (15.57 g, 67.6 mmol), 1,4-butanediol (76.5 g, 850 mmol), sodium hydrogensulfate monohydrate (0.97 g, 7.0 mmol) and toluene (35 ml) was heated to 80° C. for 17 h. After cooling to room temperature the mixture was extracted with 5% aqueous sodium chloride (75 ml) and 10% aqueous sodium bicarbonate (60 ml). The organic layer was then dried over anhydrous sodium sulfate, filtered and evaporated to dryness to give 15.0 g of red oil. Chromatography on silica gel eluting with ethyl acetate and heptane (gradient 1:8, 3×1:4, 1:2) followed by vacuum drying gave 4.2 g (69%) of (S)-naproxen 4-hydroxybutyl ester having a purity of at least 98% according to HPLC. ¹H NMR (CDCl₃, TMS) δ 7.68 (app d, J=8 Hz, 2 H), 7.64 (app br s, 1 H), 7.38 (app br d, J=8 Hz, 1 H), 7.06-7.16 (m, 2 H), 4.07 (app t, J=6 Hz, 2 H), 3.86 (s, 3 H), 3.83 (q, J=7 Hz, 1 H), 3.50 (app t, J=6 Hz, 2 H), 2.15 (app br s, 1 H), 1.52-1.68 (m, 2 H), 1.54 (d, J=7 Hz, 3 H), 1.40-1.52 (m, 2H); ¹³C NMR (CDCl₃) δ 174.7, 157.5, 135.6, 133.5, 129.1, 128.8, 127.0, 126.1, 125.8, 118.8, 105.4, 64.5, 61.9, 55.1, 45.3, 28.8, 24.8, 18.3. MS [M+NH₄]⁺320.

### EXAMPLE 2

### (S)-Naproxen 4-hydroxybutyl Ester, Large Scale Procedure Using Purification by Extraction (S)-Naproxen (5.0 kg, 21.7 mol) was mixed with 1,4-butanediol (19.6 kg, 217 mol) and the stirred mixture was heated to 80° C. Sulfuric acid (42.5 g, 433 mmol) was added and the resulting reaction mixture was stirred at 80° C. for 6.5 h. After cooling to 50° C. toluene (3.3 kg), water (3.5 kg) and hexanes (6.7 kg) were added and the resulting two-phase system was stirred for 27 min. The aqueous layer was separated from the organic layer. Toluene (2.5 kg) and hexanes (2.5 kg) were added to the aqueous layer at 50° C. and the resulting two-phase system was stirred for 15 min before phase separation. This latter extraction of the aqueous layer was repeated twice using the same amounts of toluene (2.5 kg) and hexanes (2.5 kg). Toluene (13.0 kg) and 0.2 M potassium carbonate (aq) (14.9 kg) were added to the aqueous layer at 50° C. and the resulting two-phase system was stirred for 25 min before phase separation. Water (14.9 kg) was added to the organic layer at 50° C. and the resulting two-phase system was stirred for 15 min before phase separation. This latter extraction of the organic layer was repeated using water (14.8 kg) and after phase separation the volume of the organic layer was reduced by vacuum distillation down to a concentration of 54.7% (w/w) of the product in toluene. The isolated yield calculated from this solution was 5.6 kg (83%) of pure (S)-naproxen 4-hydroxybutyl ester uncorrected for unconsumed starting material (2.7% by area of the (S)-naproxen was unconsumed at the time of quenching) having a 1,4-butanediol-content of 0.2% (w/w) and a water content below 0.1% (w/w). The chromatographic purity was 99.5% by area. This solution was used without further treatment in the synthesis of (S)-naproxen 4(methanesulfonyloxy)-butyl ester (Example 6 below).

### EXAMPLE 3

### (S)-Naproxen 4-hydroxybutyl Ester, Procedure Using Purification by Extraction

(S)-Naproxen (200 g, 0.869 mol) was mixed with 1,4-butanediol (783 g, 8.69 mol) and the stirred mixture was heated to 80° C. Sulfuric acid (4.0 g, 40 mmol) was added and the resulting reaction mixture was stirred at 80° C. for 3 h 50 min after which >96% conversion had been reached according to LC. Toluene (218 ml) was charged followed by water (130 ml) and hexanes (312 ml) which made the inner temperature to go down to 50° C. and the resulting two-phase system was stirred for 10 min before phase separation. 1,4-Butanediol (100 ml) was added to the organic layer at 50° C. and the resulting two-phase system was stirred for 5 min before phase separation. The 1,4-butanediol-layer was added to the aqueous layer and the toluene-layer was reextracted with 1,4-butanediol (100 ml). The second 1,4-butanediol-layer was added to the aqueous layer and the combined aqueous layer was extracted with a mixture of toluene (110 ml) and hexanes (156 ml) at 50° C. After phase separation the organic layer was extracted twice with 1,4-butanediol (2×50 ml) at 50° C. and the 1,4-butanediol-layer were added to the aqueous layer. The combined aqueous layer was extracted between toluene (700 ml) and 0.2 M potassium carbonate (aq) (800 ml) at 50° C. with stirring for 10 min before phase separation. The organic layer was extracted twice with water (600 ml) at 50° C. and the organic layer was then distilled to dryness under vacuum. This gave 225 g (95%) of (S)-naproxen 4-hydroxybutyl ester having a chromatographic purity of 99.2%. The yield is corrected for LC response factors and unconsumed starting material (3.26% by area of the (S)-naproxen was unconsumed at the time of quenching). (S)-Naproxen 4-hydroxybutyl ester was used without further treatment in the synthesis of (S)-naproxen 4-(methanesulfonyloxy)butyl ester (Example 4 below).

### Synthesis of Sulfonate Esters Suitable as Intermediates for the Preparation of (S)-naproxen 4-nitrooxybutyl Ester (IV)

### EXAMPLE 4

### (S)-Naproxen 4-(4-toluenesulfonyloxy)butyl Ester, Using Purification by Chromatography

(S)-Naproxen 4-hydroxybutyl ester (7.33 g, 24.2 mmol) was mixed with toluene (40 ml) under stirring at room temperature. To this mixture were added 4-toluenesulfonyl chloride (6.01 g, 31.5 mmol), 4-(dimethylamino)pyridine (0.30 g, 2.4 mmol) and triethylamine (6.7 ml, 48.5 mmol) also at room temperature. The resulting mixture was heated at 40° C. bath temperature for 7 h after which more 4-toluenesulfonyl chloride (0.92 g, 4.8 mmol) was added. The reaction was then continued for another 18 h at 40° C. bath temperature. After allowing the mixture to cool to room temperature, 10% aqueous hydrochloric acid (30 ml) was added under stirring. Stirring was continued for about 15 min when the two phases were separated and the toluene-layer was extracted with 10% aqueous sodium bicarbonate. The toluene-layer was then dried over anhydrous sodium sulfate, filtered and evaporated to dryness to give yellow oil. Chromatography on silica gel eluting with ethyl acetate/iso-hexane (stepwise gradient 2×1:8, 2×1:6, 3×1:4 and 3×1:2) gave 9.80 g (89% yield) of pure title compound as transparent oil after vacuum drying at 30° C. over night. ¹H NMR (CDCl₃) δ 7.74 (app d, J=8 Hz, 2 H), 7.69 (app d, J=8 Hz, 2 H), 7.63 (app br s, 1 H), 7.36 (app br d, J=8 Hz, 1 H), 7.30 (app d, J=8 Hz, 2 H), 7.08-7.18 (m, 2 H), 3.98-4.60 (m, 2 H), 3.87-3.97 (m, 3 H), 3.92 (s, 3 H), 2.43 (s, 3 H), 1.50-1.66 (m, 4 H), 1,55 (d, J=7 Hz, 3 H); ¹³C NMR (CDCl₃) δ 174.9, 158.1, 145.2, 136.0, 134.1, 133.4, 130.3, 129.6, 129.3, 128.2, 127.6, 126.6, 126.3, 119.4, 106.0, 70.2, 64.1, 55.7, 45.8, 25.8, 25.1, 22.0, 18.8. MS [M+NH₄]⁺474.

### EXAMPLE 5

### (S)-Naproxen 4-(methanesulfonyloxy)butyl Ester, Using Purification by Crystallisation

(S)-Naproxen (4-hydroxybutyl)ester (8.37 g, 27.7 mmol), me thanesulfonyl chloride (3.68 g, 32.1 mmol) and toluene (40 ml) were charged to a three-necked round bottomed flask. The mixture was stirred and to the resultant clear solution was added triethylamine (3.07 g, 30.3 mmol), drop-wise from the dropping funnel the temperature increasing to 60° C. during the addition. Precipitation of a white solid occurred which made stirring difficult so toluene (40 ml) was added to facilitate stirring. Upon completion of addition, the reaction mixture stirred at room temperature for 3 days. 1 M HCl (30 ml) was added and the mixture was heated to 60° C. in order to dissolve the solids present. Upon dissolution of the solids, the mixture was decanted to a separating funnel and the two phases separated whilst still hot n-Heptane (50 ml) was added slowly to the organic phase, maintaining the temperature at 50° C., causing some precipitation to occur. This mixture was allowed to cool to room temperature and was left standing at room temperature for 3 hours. The precipitated product was collected by filtration and the solids were washed with n-heptane (50 ml) and then dried at 30° C. under vacuum. Yield 8.52 g (81%) of a white solid. HPLC purity >95%. ¹H NMR (CDCl₃) δ 7.68 (app d, J=8 Hz, 2 H), 7.65 (app br s, 1 H), 7.39 (app br d, J=8 Hz, 1 H), 7.08-7.18 (m, 2 H), 4.06-4.16 (m, 4 H), 3.90 (s, 3 H), 3.84 (q, J=7 Hz, 1 H), 2.88 (s, 3 H), 1.62-1.74 (m, 4 H), 1.57 (d, J=7 Hz, 3 H); ¹³C NMR (CDCl₃) δ 174.5, 157.6, 135.6, 133.6, 129.2, 128.8, 127.1, 126.1, 125.8, 119.0, 105.5, 69.2, 63.7, 55.2, 45.4, 37.2, 25.7, 24.7, 18.3. MS [M+NH₄]+398.

### EXAMPLE 6

### (S)-Naproxen 4-(methanesulfonyloxy)butyl Ester, Large Scale Procedure Using Purification by Crystallisation

Toluene (24.6 kg) was added to the obtained solution of (S)-naproxen 4-hydroxybutyl ester from Example 2 (5.6 kg, 18.5 mol mixed with 4.6 kg of toluene) and the resulting solution was stirred and heated to 35° C. Methanesulfonyl chloride (2.6 kg, 22.7 mol) was added over 1 min and the addition vessel was rinsed with toluene (6.1 kg divided in two portions) letting the washing phase to go into the reaction mixture. N-Methylmorpholine (2.1 kg, 20.8 mol) was added over 54 min with efficient stirring during which the inner temperature increased from 38.5° C. to 45.8° C. The addition vessel was rinsed with toluene (3.1 kg divided in two portions) letting the washing phase to go into the reaction mixture. The reaction mixture was then stirred for 4 h at 43° C. before raising the temperature to 58° C. When this temperature had been reached 0.1 M. sulfuric acid (22.4 kg) was added and the resulting two-phase system was stirred at 58° C. for 22 min before phase separation. The organic layer was mixed with water (22.4 kg) and the resulting two-phase system was stirred at 58° C. for 16 min before phase separation and the temperature of the organic layer was then raised to 61° C. Cooling of the organic layer to 3° C. over 5 h 15 min gave a slurry which was filtered. The obtained solid was washed with toluene (9.8 kg) at 20° C. and the filter cake was blown dry with nitrogen for about 60 min. The obtained crystals were recrystallized according to the same procedure using toluene (31 kg) and after washing the filtered crystals with toluene (9.7 kg) at 20° C. and drying under vacuum at 36° C. for 20 h 5.0 kg (71%) of pure (S)-naproxen 4-(methanesulfonyl-oxy)butyl ester having a chromatographic purity of 99.5%, an assay of 97.6% (w/w) and an optical purity of 99.8% enantiomeric excess was obtained.

### EXAMPLE 7

### (S)-Naproxen 4-(methanesulfonyloxy)butyl Ester, Using Purification by Crystallisation

(S)-Naproxen 4-hydroxybutyl ester (200 g, 0.661 mol) and toluene (1.40 L) were mixed and the resulting solution was stirred and heated to 40° C. Methanesulfonyl chloride (85.0 g, 22.7 mol) was added and the mixture was stirred for 20 min at 40° C. N-Methylmorpholine (71.7 g, 0.709 mol) was added over 10 min with efficient stirring. The reaction mixture was then stirred for 3 h 15 min at 40° C. before another portion of N-methylmorpholine (6.8 g, 0.067 mol) was added. After stirring for another 60 min at 40° C. another portion of N-methylmorpholine (3.3 g, 0.033 mol) was added and the reaction was allowed to continue for another 80 min (total reaction time 4 h 35 min). The reaction was then complete according to LC and 0.1 M sulfuric acid (700 ml) was added. The resulting two-phase system was stirred at 40° C. for 15 min before phase separation. The organic layer was mixed with water (700 ml) and the resulting two-phase system was stirred during heating to 60° C. for 40 min before another portion of toluene (100 ml) and water (200 ml) was added. Stirring at 60° C. was continued for another 15 min before phase separation. The volume of the organic layer was then reduced by vacuum distillation at a jacket temperature of 60° C. so that approximately half of the toluene (about 600 ml) remained. n-Heptane (800 ml) was added and after cooling from an inner temperature of 53° C. to 4° C. over 2 h a slurry was obtained which was filtered. The isolated solid including remaining solids in the reactor was washed with n-heptane (1.0 L) and dried under vacuum at 40° C. for 7 h giving 232 g (92%) of (S)-naproxen 4-(methanesulfonyloxy)butyl ester having a chromatographic purity of 98.6%.

### EXAMPLE 8

### (S)-Naproxen 4-(benzylsulfonyloxy)butyl Ester

(S)-Naproxen 4hydroxybutyl ester (10.0 g, 33.1 mmol) and □-toluenesulfonyl chloride (7.6 g, 40 mmol) were mixed with ethyl acetate (50 ml) and the resulting solution was stirred at room temperature. N-Methylmorpholine (3.7 g, 36.6 mmol) was added over 10 min under stirring. The reaction mixture was then stirred for 30 min at room temperature before another portion of N-methylmorpholine (0.33 ml, 3.0 mmol) was added. After stirring for another 25 min at room temperature 50 ml of 0.10 M sulphuric acid was added. After extraction and phase separation the organic layer was washed with water (50 ml). The volume of the organic layer was reduced to about 15 ml by vacuum distillation and the temperature was then increased to 60° C. n-Heptane (20 ml) was added and the temperature was decreased to 0° C. over 1 h before filtration of the solid. The crystals were rinsed with n-heptane (10 ml) and dried under vacuum at 40° C. to give 14.1 g (97%) of (S)-naproxen 4-(benzylsulfonyloxy)butyl ester having a chromatographic purity of 99%. ¹H NMR (CDCl₃) δ 7.72 (app d, J=8.6 Hz, 2 H), 7.68 (app d, J=1.3 Hz, 1 H), 7.31-7.42 (m, 6 H), 7.16 (app dd, J1=8.6 Hz, J2=2.5 Hz, 1 H), 7.13 (app d, J=2.5 Hz, 1 H), 4.28 (s, 2 H), 4.03-4.09 (m, 2 H), 3.89-4.01 (m, 2 H), 3.93 (s, 3 H), 3.86 (q, J=7.1 Hz, 1 H), 1.56-1.62 (m, 7 H).

### EXAMPLE 9

### (S)-Naproxen 4-(4-chlorobenzenesulfonyloxy)butyl Ester

(S)-Naproxen 4-hydroxybutyl ester (10.0 g, 33.1 mmol) was mixed with acetonitrile (40 ml) and the resulting solution was cooled down to 5° C. under stirring. 4-Chlorobenzenesulfonyl chloride (10.4 g, 49.1 mmol) was added and to the resulting solution at 5° C. N-methylmorpholine (4.3 ml, 39.1 mmol) was added with efficient stirring. A white precipitate gradually formed and the reaction mixture was stirred over night at 5° C. after which LC showed 91% conversion. Dilute sulphuric acid (0.1 M, 20 ml) was added and the resulting slurry was stirred for 15 min. Ethyl acetate (40 ml) was added and the mixture was heated to 40° C. creating a biphasic mixture. The organic layer was washed with water (40 ml) and then concentrated to half its original volume using vacuum distillation. Cooling to 0° C. and stirring at that temperature for 2 h gave a slurry from which the solid was isolated by filtration. This solid was recrystallised from ethyl acetate (23 ml) using hexane (53 ml) as antisolvent. The crystals were filtered and rinsed with hexane and dried under vacuum at 40° C. to give 10.1 g (64%) of (S)-naproxen 4-(4-chlorobenzenesulfonyloxy)butyl ester having a chromatographic purity of >99%. Melting point 74-75° C.; ¹H NMR (CDCl₃) δ 7.75-7.82 (m, 2 H), 7.71 (app d, J=8.6 Hz, 2 H), 7.65 (app d, J=1 Hz, 1 H), 7.46-7.53 (m, 2 H), 7.38 (app dd, J1=8.5 Hz, J2=1.8 Hz, 1 H), 7.16 (app dd, J1=8.5 Hz, J2=2.6 Hz, 1 H), 7.12 (app d, J=2.5 Hz, 1 H), 3.98-4.08 (m, 2 H), 3.88-3.98 (m, 2 H), 3.93 (s, 3 H), 3.83 (q, J=7.2 Hz, 1 H), 1.51-1.66 (m, 7 H); ¹³C NMR (CDCl₃) δ 174.9, 158.0, 140.8, 135.9, 134.8, 134.0, 129.9, 129.5, 129.2, 127.5, 126.4, 126.2, 119.4, 105.9, 70.6, 63.9, 55.6, 45.7, 25.7, 25.0, 18.7. MS [M+NH₄]⁺494.

### Preparation of (S)-naproxen 4-nitrooxybutyl Ester

### EXAMPLE 10

### Using Purification by Chromatography

(S)-Naproxen (4-toluenesulfonyloxy)butyl ester (4.05 g, 8.87 mmol) was mixed with sodium nitrate (1.51 g, 17.7 mmol) and N-methylpyrrolidinone (NMP, 8 ml) and the mixture was heated with stirring on a 70° C. oil bath for 21 h. Water (30 ml) was added with stirring and the oily product separated out and was washed with a further 2×20 ml of water after separation from the first aqueous layer. The obtained oil was then partitioned between toluene (20+10 ml) and water (20 ml) and after phase separation the organic layer was dried using sodium sulfate, filtered and evaporated to dryness to give 2.64 g of an orange oil. The first aqueous layer above was reextracted with toluene (30+10 ml) and this second toluene layer was also dried using sodium sulfate, filtered and evaporated to dryness to give 0.47 g of a less viscous orange oil. The two oily residues were combined and purified by silica gel chromatography eluting with ethyl acetate/n-heptane (stepwise gradient 1:8, 3×1:4 and 1:2) to give 2.61 g (85% yield) of pure (S)-naproxen 4-nitrooxybutyl ester (transparent thick oil) after drying under vacuum at 30° C. over night. ¹H NMR δ 7.70 (app d, J=8 Hz, 2 H), 7.65 (app br s, 1 H), 7.38 (app br d, J=8 Hz, 1 H), 7.07-7.18 (m, 2 H), 4.26-4.36 (m, 2 H), 4.02-4.17 (m, 2 H), 3.92 (s, 3 H), 3.84 (q, J=8 Hz, 1H), 1.52-1.74 (m, 4 H), 1.58 (d,=7 Hz, 3 H); ¹³C NMR δ 175.0, 158.1, 136.0, 134.1, 129.6, 129.3, 127.6, 126.5, 126.3, 119.5, 106.0, 72.9, 64.1, 55.7, 45.9, 25.3, 23.9, 18.7. MS [M+NH₄]⁺365. The amount of (R)-enantiomer present was 3.46% as determined by chiral HPLC. The batch of (S)-naproxen used to prepare this batch and also the ones in Example 11 and 12 below contains 3.35% of the (R)-enantiomer as determined by chiral HPLC.

### EXAMPLE 11

### Using Purification by Chromatography

(S)-Naproxen (4-toluenesulfonyloxy)butyl ester (3.40 g, 7.45 mmol) and tetrabutylammonium hydrogensulfate (20 mg, 0.060 mmol) were mixed with sodium nitrate (10.6 ml of a 7.0 M aqueous solution, 74.5 mmol) and toluene (17 ml) and the resulting two-phase system was stirred at 100° C. for 6 days. After cooling to room temperature the toluene-layer was separated off and the aqueous layer was extracted with more toluene (10 ml). The two toluene-portions were combined and evaporated to dryness. Purification by chromatography on silica gel eluting with ethyl acetate/n-heptane (stepwise gradient 2×1:8, 1:6 and 3×1:4) gave 2.20 g (85% yield) of pure (S)-naproxen 4-nitrooxybutyl ester after drying under vacuum at 30° C. over night. The spectral data are the same as for Example 10 above. The amount of (R)-enantiomer present was 3.47% as determined by chiral HPLC. The batch of (S)-naproxen used to prepare this batch and also the ones in Examples 10 above and 12 below contains 3.35% of the (R)-enantiomer as determined by chiral HPLC.

### EXAMPLE 12

### Using Purification by Chromatography

(S)-Naproxen 4-nitrooxybutyl ester was prepared essentially according to Example 4 above but starting from (S)-naproxen (4-methanesulfonyloxybutyl) ester (8.33 g, 21.9 mmol), sodium nitrate (3.72 g, 43.8 mmol) and N-methylpyrrolidinone (NMP, 23 ml). The reaction temperature used was 80° C. and the reaction time 21 h. After work up using extraction between toluene and water 7.30 g of crude product was obtained. This was purified by column chromatography on silica gel eluting with heptane/ethyl acetate (4:1). Evaporation of solvent under reduced pressure gave 5.62 g (74%) of the pure product having the same spectroscopic data as for Example 4 above. The amount of (R)-enantiomer present was 1.98% as determined by chiral HPLC. Since the batch of (S)-naproxen used to prepare the intermediates for this Example and Examples 10 and 11 above contains 3.35% of the (R)-enantiomer, an improvement also with regard to optical purity has occurred during the crystallisation of (S)-naproxen 4-(methanesulfonyloxy)butyl ester described in Example 5.

### EXAMPLE 13

### Large Scale Example

(S)-Naproxen 4-(methanesulfonyloxy)butyl ester (1.6 kg, 4.2 mol) was mixed with lithium nitrate (0.90 kg, 13 mol) and N-methylpyrrolidinone (NMP) (6.8 kg) and the mixture was stirred under nitrogen between 75-78° C. for 12 h 40 min when LC showed 99.9% conversion. After the temperature had been lowered to 20-22° C. the mixture was left to stir under nitrogen for 20 h. tert-Butylmethyl ether (4.9 kg) and 0.5 M sodium bicarbonate (6.9 kg) were added and the resulting two-phase system was stirred for 16 min before phase separation. The organic layer was washed with water (6.6 kg) for 17 min before phase separation and this extraction was repeated using water (6.6 kg) to give an organic layer containing less than 0.1% (wlw) of NMP after phase separation. The organic layer was filtered to remove any solids and after vacuum distillation to dryness at a jacket temperature of between 40° C. and 46° C., 1.04 kg (73%) of (S)-naproxen 4-(nitrooxy)butyl ester was obtained as a yellow oil. The chromatographic purity was 98.6%, the assay 98.6% (wlw), the water content <0.1% (w/w), the tert-butylmethyl ether content 0.3% (w/w), the NMP content 0.1% (w/w) and the butandiol content 0.1% (w/w). The optical purity as determined using LC was 99.8% enantiomeric excess.

### EXAMPLE 14

### Large Scale Example

(S)-Naproxen 4-(methanesulfonyloxy)butyl ester (2.0 kg, 5.3 mol) was mixed with sodium nitrate (2.2 kg, 26 mol) and N-methylpyrrolidinone (NMP, 12.2 kg) and the mixture was stirred under nitrogen at 76° C. for 21.5 h before the jacket temperature was lowered to 65° C. After stirring for another 10 h at this temperature LC showed 99.9% conversion. After the temperature had been lowered to 21-22° C. the mixture was left to stir under nitrogen for 11.5 h. tert-Butylmethyl ether (5.9 kg) and 0.5 M sodium bicarbonate (8.0 kg) were added and the resulting two-phase system was stirred for 15 min before phase separation. The organic layer was washed with water (8.0 kg) for 15 min before phase separation and this extraction was repeated using water (8.0 kg), to give an organic layer containing less than 0.1% (w/w) of NMP after phase separation. The organic layer was filtered to remove any solids and after vacuum distillation to dryness at a jacket temperature of between 43° C. and 1.43 kg (77%) of (S)-naproxen 4-(nitrooxy)butyl ester was obtained as a yellow oil. The chromatographic purity was 98.4%, the assay 97.8% (w/w), the water content 0.1% (wlw), the tert-butylmethyl ether content 0.3% (w/w), the NMP content 0.2% (w/w) and the butandiol content 0.1% (w/w). The optical purity as determined using LC was 99.8% enantiomeric excess.

### EXAMPLE 15

A mixture of (S)-naproxen 4-(methanesulfonyloxy)butyl ester (3.03 g, 7.96 mmol), lithium nitrate (1.70 g, 24.7 mmol) in sulpholane (12 ml) was heated under stirring at 83° C. for 31 h when LC showed >99.5% conversion. The reaction mixture was allowed to cool down to room temperature before it was partitioned between water (24 ml) and tert-butyl methyl ether (24 ml). The organic phase was washed with water (24 ml) and evaporated to dryness under vacuum. This gave an oil containing about 14% of sulpholane and the oily product was consequently redissolved in tert-butyl methyl ether (25 ml). Washing of this solution twice with water (25 ml), drying of the organic layer using magnesium sulfate and removal of the volatiles in vacuo gave a yellow oil which was further dried at 46° C. under vacuum to give 2.06 g (74%) of (S)-naproxen 4-nitrooxybutyl ester having a chromatographic purity of 98% according to LC. ¹H-NMR-data was in accordance with the data obtained in Example 10 above.

### EXAMPLE 16

A mixture of (S)-naproxen 4-(methanesulfonyloxy)butyl ester (2.98 g, 7.83 mmol), lithium nitrate (1.64 g, 23.8 mmol) in tetramethylurea (12 ml) was heated under stirring at 83° C. for 7 h 30 min when LC showed 99.8% conversion. The reaction mixture was partitioned between water (25 ml) and tert-butyl methyl ether (25 ml). The organic phase was washed three times with water (25 ml) and evaporated to dryness under vacuum. This gave an oil which was further dried at 50° C. under vacuum to give 2.43 g (89%) of (S)-naproxen 4-nitrooxybutyl ester having a chromatographic purity of 99% according to LC. ¹H-NMR-data was in accordance with the data obtained in Example 10 above.

### EXAMPLE 17

A mixture of (S)-naproxen 4-(benzylsulfonyloxy)butyl ester (1.0 g, 2.2 mmol) and lithium nitrate (0.72 g, 10 mmol) in 1,3-dimethylimidazolidin-2-one (4.0 ml) was heated under stirring at 80° C. for 8 h when LC showed full conversion. The reaction mixture was partitioned between water (12 ml) and tert-butyl methyl ether (12 ml). The organic phase was washed with water (12 ml) and evaporated to dryness under vacuum. Drying of the oil at 40° C. under vacuum gave 0.75 g (99%) of (S)-naproxen 4-nitrooxybutyl ester having a chromatographic purity of >97% according to LC. ¹H-NMR-data was in accordance with the data obtained in Example 10 above.

### EXAMPLE 18

A mixture of (S)-naproxen 4-(benzylsulfonyloxy)butyl ester (1.0 g, 2.2 mmol), lithium nitrate (0.45 g, 6.5 mmol) and tetrabutylammonium nitrate (0.20 g, 0.66 mmol) in ethyl acetate (4.0 ml) was heated under stirring at 80° C. for 23 h when LC showed full conversion. The reaction mixture was partitioned between water (12 ml) and tert-butyl methyl ether (12 ml). The organic phase was washed with water (12 ml) and evaporated to dryness under vacuum. Drying of the oil at 40° C. under vacuum gave 0.75 g (99%) of (S)-naproxen 4-nitrooxybutyl ester having a chromatographic purity of 99% according to LC. ¹H-NMR-data was in accordance with the data obtained in Example 10 above.

### EXAMPLE 19

A mixture of (S)-naproxen 4-(benzylsulfonyloxy)butyl ester (1.0 g, 2.2 mmol) and lithium nitrate (0.45 g, 6.5 mmol) in tetramethylurea (4.0 ml) was heated under stirring at 80° C. for 21 h when LC showed full conversion. The reaction mixture was partitioned between water (12 ml) and tert-butyl methyl ether (12 ml). The organic phase was washed with water (12 ml) and evaporated to dryness under vacuum. Drying of the oil at 40° C. under vacuum gave 0.75 g (99%) of (S)-naproxen 4-nitrooxybutyl ester having a chromatographic purity of 99% according to LC. ¹H-NMR-data was in accordance with the data obtained in Example 10 above.

### EXAMPLE 20

A mixture of (S)-naproxen 4-(4-chlorobenzenesulfonyloxy)butyl ester (2.0 g, 4.2 mmol), sodium nitrate (1.07 g, 12.6 mmol) in tetramethylurea (5.0 ml) was heated under stirring at 70° C. for 2 h and at 80° C. for 5 h 40 min. The reaction mixture was cooled down to room temperature and was then partitioned between water (10 ml) and tert-butyl methyl ether (10 ml). The organic phase was washed twice with water (10 ml) and evaporated to dryness under vacuum. Drying of the oil at 40° C. under vacuum gave 1.40 g (96%) of (S)-naproxen 4-nitrooxybutyl ester having a chromatographic purity of >99% according to LC. ¹H-NMR-data was in accordance with the data obtained in Example 10 above.

### EXAMPLE 21

A mixture of (S)-naproxen 4-(4-chlorobenzenesulfonyloxy)butyl ester (2.0 g, 4.2 mmol) and lithium nitrate (0.58 g, 8.4 mmol) in 1,3-dimethylimidazolidin-2-one (5.0 ml) was heated under stirring at 70° C. for 3 h. The reaction mixture was partitioned between water. (5 ml) and tert-butyl methyl ether (5 ml). The organic phase was washed twice with water (5 ml) and evaporated to dryness under vacuum. Drying of the oil at 40° C. under vacuum gave 1.35 g (93%) of (S)-naproxen 4-nitrooxybutyl ester having a chromatographic purity of 98% according to LC. ¹H-NMR-data was in accordance with the data obtained in Example 10 above.

### EXAMPLE 22

A mixture of (S)-naproxen 4-(4-chlorobenzenesulfonyloxy)butyl ester (2.0 g, 4.2 mmol), lithium nitrate (0.87 g, 13 mmol) in sulpholane (5.0 ml) was heated under stirring at 70° C. for 2 h 50 min and at 80° C. for 6 h 20 min and then at 60° C. for 15 h. The reaction mixture was cooled down to room temperature and was then partitioned between water (10 ml) and tert-butyl methyl ether (10 ml). The organic phase was washed twice with water (10 ml) and evaporated to dryness under vacuum. Drying of the oil at 40° C. under vacuum gave 1.38 g (95%) of (S)-naproxen 4-nitrooxybutyl ester having a chromatographic purity of >99% according to LC. ¹H-NMR-data was in accordance with the data obtained in Example 10 above.

### EXAMPLE 23

A mixture of (S)-naproxen 4-(benzylsulfonyloxy)butyl ester (1.0 g, 2.2 mmol), sodium nitrate (0.37 g, 4.4 mmol) and tetrabutylammonium nitrate (0.07 g, 0.2 mmol) in methyl iso-butyl ketone (2 ml) was heated under stirring at 80° C. for 18 h 30 min when LC showed full conversion. The reaction mixture was diluted with methyl iso-butyl ketone (2 ml) and the organic phase was washed three times with water (4 ml) and evaporated to dryness under vacuum. This gave 0.73 g (96%) of (S)-naproxen 4-nitrooxybutyl ester having a chromatographic purity of 98% according to LC. ¹H-NMR-data was in accordance with the data obtained in Example 10 above.

### EXAMPLE 24

A mixture of (S)-naproxen 4-(methanesulfonyloxy)butyl ester (1.0 g, 2.6 mmol), sodium nitrate (0.65 g, 7.6 mmol) and tetrabutylammonium nitrate (0.13 g, 0.43 mmol) in ethyl acetate (4 mL) was heated under stirring at 80° C. for 26 h when LC showed full conversion. The reaction mixture was partitioned between teri-butyl methyl ether (5 mL) and water (5 mL) and the organic layer was washed three times with water (5 mL) before evaporation to dryness under vacuum. This gave 0.77 g (84%) of (S)-naproxen 4-nitroxybutyl ester having a chromatographic purity of 99% according to LC. ¹H-NMR-data was in accordance with the data obtained in Example 10 above.

### EXAMPLE 25

A mixture of (S)-naproxen 4-(methanesulfonyloxy)butyl ester (1.0 g, 2.6 mmol), sodium nitrate (0.68 g, 8.0 mmol) and tetrabutylammonium nitrate (0.13 g, 0.43 mmol) in methyl iso-butyl ketone (4 mL) was heated under stirring at 80° C. for 17 h 40 min when LC showed full conversion. The reaction mixture was partitioned between tert-butyl methyl ether (5 mL) and water (5 mL) and the organic layer was washed three times with water (5 mL) before evaporation to dryness under vacuum. This gave 0.75 g (82%) of (S)-naproxen 4-nitroxybutyl ester having a chromatographic purity of 99% according to LC. ¹H-NMR-data was in accordance with the data obtained in Example 10 above.

1. A process for the preparation of (S)-naproxen 4-nitrooxybutyl ester (IV) 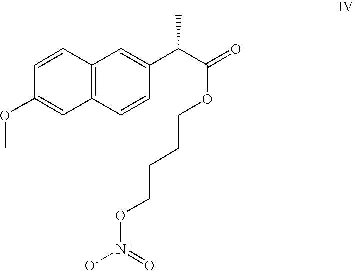
comprising:
1a) reacting (S)-naproxen, or an acid halide or a salt derivative of (S)-naproxen, 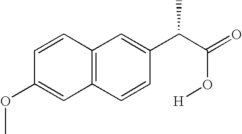
with a compound of formula II, 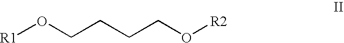
wherein R1 is H or RSO₂, and R2 is RSO₂, and
R is C₁-C₄ alkyl, phenyl, phenylmethyl, C₁-C₄ alkylphenyl, halophenyl, nitrophenyl, halogen, CF₃ or n-C₄F₉, and the halo or halogen is fluoro, chloro or bromo,
to obtain a compound of formula I, 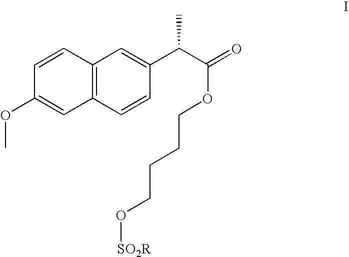
and
2) reacting the compound of formula I with a nitrate source, optionally in the presence of a solvent, to obtain (S)-naproxen 4-nitrooxybutyl ester (IV).

2. A process for the preparation of (S)-naproxen 4-nitrooxybutyl ester (IV) 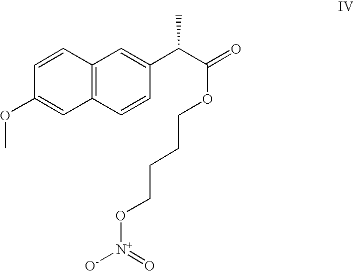
comprising:
1b) reacting (S)-naproxen with a compound of formula II, 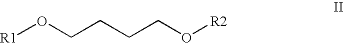
wherein R1 and R2 are both H, to obtain a compound of formula III, 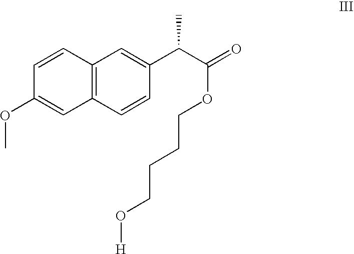
and thereafter,
1c) reacting the compound of formula III with RSO₂Cl to give a compound of formula I, 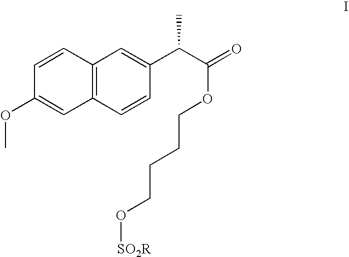
wherein
R is C₁-C₄ alkyl, phenyl, phenylmethyl, C₁-C₄ alkylphenyl, halophenyl, nitrophenyl, halogen, CF₃ or n-C₄F₉, and the halo or halogen is fluoro, chloro or bromo,
and
2) reacting the compound of formula I with a nitrate source, optionally in the presence of a solvent, to obtain (S)-naproxen 4-nitrooxybutyl ester.

3. The process according to claim 1 or 2, wherein R is C₁-C₄ alkyl, C₁-C₄ alkylphenyl, halophenyl, nitrophenyl, halogen, CF₃ or n-C₄F₉, and the halo or halogen is fluoro, chloro or bromo.

4. The process according to claim 2, whereby wherein step 1b is performed in the presence of an acidic agent or dehydrating agent.

5. The process according to claim 4, wherein the acidic agent or dehydrating agent 1b is selected from the group consisting of sulphuric acid, a salt thereof, perchloric acid, polystyrene sulphonic acids, zeolites, acidic clays, sand/strong hydrophilic acid combinations, and montmorillonites.

6. The process according to claim 4, wherein the solvent in step 1b is selected from the group consisting of aromatic hydrocarbons, aliphatic hydrocarbons, ketones, ethers, chlorinated hydrocarbons, and mixtures thereof.

7. The process according to claim 4, wherein the solvent in step 1b comprises an excess of 1,4-butanediol, optionally mixed with one or more solvents selected from the group consisting of aromatic hydrocarbons, aliphatic hydrocarbons, ketones, ethers, chlorinated hydrocarbons, and mixtures thereof.

8. The process according to claim 4, wherein the compound of formula III is purified by way of a two-way extraction either batch-wise or continuously to obtain a solution comprising a compound of formula III having a chromatographic purity of at least 95% and a 1,4-butanediol concentration below about 0.2% (w/w).

9. The process according to claim 8, wherein the extraction solution of the first extraction step comprises a mixture of i) 1,4-butanediol, ii) water and/or a low molecular weight aliphatic alcohol and iii) a hydrocarbon solvent or mixtures of organic solvents with hydrocarbon solvents.

10. The process according to claim 2, wherein step 1c is performed in the presence of a base and optionally in the presence of a catalyst.

11. The process according to claim 10, wherein the base is selected from the group consisting of N-methylmorpholine, triethylamine, pyridine, diisopropylethylamine, tributylamine, and N-methyl-piperidine.

12. The process according to claim 10, wherein the catalyst is 4-(dimethylamino)-pyridine.

13. The process according to claim 2, wherein the solvent in step 1c is selected from the group consisting of aromatic hydrocarbons, ketones, ethers, aliphatic nitriles, and aliphatic esters, and mixtures thereof.

14. The process according to claim 1 or 2, wherein the compound of formula I is purified by crystallisation from an organic solvent, optionally using a hydrocarbon as an antisolvent to obtain a crystalline solid.

15. The process according to claim 1 or 2, wherein the nitrate source is selected from the group consisting of lithium nitrate, sodium nitrate, potassium nitrate, magnesium nitrate, and calcium nitrate.

16. The process according to claim 1 or 2, wherein the solvent in step 2 is an organic solvent selected from the group consisting of aromatic hydrocarbons, aliphatic hydrocarbons, ketones, ethers, chlorinated hydrocarbons, nitrites, aliphatic esters, polar aprotic solvents, nitrated hydrocarbons, ethylene glycols, and mixtures thereof, optionally with an added aliphatic alcohol.

17. The process according to claim 1 or 2, wherein the solvent in step 2 is water, optionally in combination with one or more organic solvents selected from the group consisting of aromatic hydrocarbons, aliphatic hydrocarbons, ketones, ethers, chlorinated hydrocarbons, nitriles, aliphatic esters, polar aprotic solvents, nitrated hydrocarbons, ethylene glycols, and mixtures thereof, optionally with an added aliphatic alcohol.

18. The process according to claim 1 or 2, wherein the solvent in step 2 is water and/or an organic solvent selected from the group consisting of N-methylpyrrolidinone, sulpholane, tetramethylurea, 1,3-dimethyl-2-imidazolidinone, ethyl acetate, butyl acetate, isopropyl acetate, methyl ethyl ketone, methyl isobutyl ketone, acetonitrile, and mixtures thereof.

19. The process according to claim 1 or 2, wherein step 2 is performed in the presence of a phase-transfer-catalyst.

20. The process according to claim 1 or 2, wherein step 2 is performed in the presence of a detergent selected from the group consisting of a non-ionic surfactant, an ionic surfactant, and mixtures thereof.

21. The process according to claim 1 or 2, wherein the process is performed at a temperature in the range of about −100° C. to about +130° C.

22. A compound of formula I 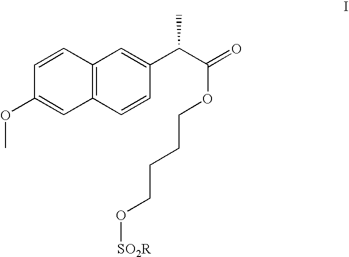
wherein R is phenylmethyl, halophenyl, nitrophenyl, halogen, CF₃ or n-C₄F₉ and the halo or halogen is fluoro, chloro or bromo

23. The compound according to claim 22, wherein R is halophenyl, nitrophenyl, halogen, CF₃ or n-C₄F₉ and the halo or halogen is fluoro, chloro or bromo

24-26. (canceled)

27. The process according to claim 1 or 2, wherein the process is a large-scale production of (S)-naproxen 4-nitrooxybutyl ester.

28. (canceled)
